(19) United States
(12) Patent Application Publication
(10) Pub. No.: US 2026/0272593 A1
(43) Pub. Date: Sep. 17, 2026

(54) APPARATUS AND METHODS FOR HEMODYNAMIC MONITORING

(71) Applicant: Chirag Rajyaguru, Carmel, IN (US)

(72) Inventor: Chirag Rajyaguru, Carmel, IN (US)

(21) Appl. No.: 19/566,385

(22) Filed: Mar. 13, 2026

Related U.S. Application Data
(60) Provisional application No. 63/771,057, filed on Mar. 13, 2025.
(60) Provisional application No. 63/825,111, filed on Jun. 17, 2025.

(51) Int. Cl. A61B 50/20; A61B 5/00; A61B 90/00

(52) U.S. Cl. CPC A61B 50/20; A61B 5/6823; A61B 5/6831; A61B 5/6833; A61B 90/08; A61B 2090/0808

(57) ABSTRACT

Described herein are medical device holders for holding and maintaining a position of one or more transducers relative to a body of a patient. The medical device holders include a base structure configured to be applied to the body of the patient and one or more transducer holders coupled to the base structure. Each transducer holder is configured to retain a respective transducer therein. The medical device holders also include a mid-line indicator configured to align with a mid-axillary line of the patient when the medical device holder is applied to the body of the patient.

### CROSS-REFERENCE TO RELATED APPLICATIONS

This application claims the benefit of priority to U.S. Provisional Patent Application No. 63/771,057, filed Mar. 13, 2025, and to U.S. Provisional Patent Application No. 63/825,111, filed Jun. 17, 2025, the entire contents of each of which are hereby incorporated by reference herein.

### FIELD OF DISCLOSURE

The present disclosure relates generally to an apparatus for improved hemodynamic monitoring, such as a medical device holder, and methods related thereto. In particular, the present disclosure relates to transducer holders configured to maintain a position of one or more transducers on the body of a patient.

### BACKGROUND

Patients admitted to the hospital who are critically ill are admitted to the intensive care unit (ICU) as a standard of practice. These patients are often unstable and require monitoring of their hemodynamics. This monitoring can be performed using an invasive arterial line or a pulmonary artery catheter (PAC).

Currently, arterial lines involve placement of a sheath in an arterial vessel. Once the catheter is inserted, it is connected to a transducer, which converts blood pressure readings from the area of interest into an electrical signal. The transducer is connected to a monitor which displays this electrical signal. A pressure bag with a saline solution attached to the transducer flushes the tubing of unwanted components (air, blood, etc.). During the monitoring phase, it is important that the transducer be placed at the level of the right atrium and, frequently, in a position halfway between the anterior chest wall and the surface on which the patient is laid (that is, at the mid-axillary line). At this placement, the pressure transducer is zeroed to atmospheric pressure. Although transducer placement location can vary by practice, it is recognized that the transducer should be at the level of the vessel for which measurements are being captured. Any significant alteration in this level can lead to inaccurate measurements.

A PAC procedure is a venous procedure. Hemodynamic monitoring from this type of catheter may include monitoring of right atrial, pulmonary artery, and pulmonary capillary wedge pressures, as well as monitoring of cardiac output. This catheter has multiple ports, including a distal tip resting in the pulmonary artery and a proximal port resting in the right atrium. Both ports are continuously monitored. Similar to the arterial line procedure, the catheter ports are connected to a transducer, which will convert the mechanical pressure signals into electrical signals. A connected monitor displays the electrical signals. Also similar to the arterial line procedure, the level of transducer should be the same as the level of the right atrium.

As these procedures are occurring more frequently, common sources of error need to be addressed to improve the quality of data that can be provided by these potentially lifesaving tools. One of the most common sources of error involves zeroing of the transducer, specifically when the transducer is incorrectly positioned. Currently, the most common tool used to position and zero a transducer includes a transducer holder attached to an IV pole. The transducer holder is placed at the level of the right atrium using a long straight edge (e.g., a ruler, stick, etc.) that is held at and extended from the mid-thoracic level. This method can be very accurate and reproducible, if the time is taken to do it correctly. However, this method has many limitations. It takes a significant amount of time to lay the patient flat, ensure the hospital bed (or other surface) is also flat, access a suitable straight edge, and carefully perform the leveling placement, which must be repeated each time new hemodynamic values are obtained. Moreover, any patient movement or change in position of the hospital bed necessitates restarting the process.

Alternate methods have been attempted. For example, medical tape may be used to secure transducers to a patient's arm or chest wall (rather than the IV pole). However, this method is relatively unreliable, because transducers are not often taped at the correct level, and the tape frequently needs to be reapplied.

An arm-worn transducer holder is also available, but this placement method can also lead to significant inaccuracies. For example, these known arm-worn holders position the transducers in parallel, so only one transducer is appropriately positioned at the mid-axillary line; indeed, in such an arrangement, it is not physically possible for additional transducers to be at the appropriate level. Moreover, differences in patient arm size lead to a large variability in transducer level. Still further, this apparatus may slide up and down the arm, such that the transducer level will easily change, causing measurement to become inaccurate. This is especially apparent when performing exercise hemodynamic studies in more stable patients that are being evaluated for advanced therapies such as heart transplant. For example, patients may operate a semi-upright exercise bike that results in substantial arm movement during use.

Another source of error related to hemodynamic monitoring occurs with respiratory variation, such as in patients who are obese, have lung disease, or who exercise (e.g., during a hemodynamic study). To get accurate measurements in these patients, an esophageal pressure-transducing balloon catheter can be used, but may be uncomfortable to the patient and is often not available. Alternatively, providers may ask the patient to hold their breath, to remove respiratory variation during readings, but this practice can lead to inaccurate measurements depending on which phase of the breathing cycle is being experienced during the breath-holding. Measurements should be taken at end of expiration, as that is when intra-thoracic pressure is closest to zero, but patients may not wait until expiration is complete or may forcefully expire, both of which will affect readings.

### SUMMARY OF THE DISCLOSURE

The present disclosure is directed to a medical device holder, specifically a transducer holder, embodied on various base structures. The medical device holder ensures proper placement of the transducer during leveling and also maintains the position of the transducer for accurate pressure measurements, thereby overcoming the disadvantages described above. The medical device holder described herein also moves with the patient (e.g., during exercise studies), allowing the patient to participate in active and/or upright activities while still securing the position of the medical device holder relative to the patient.

In one embodiment, a medical device holder for holding and maintaining a position of one or more transducers relative to a body of a patient is provided. The medical device holder includes a base structure configured to be applied to the body of the patient and one or more transducer holders coupled to the base structure, each transducer holder configured to retain a respective transducer therein. The medical device holder also includes a mid-line indicator that bisects at least one of the one or more transducer holders, wherein the mid-line indicator is configured to align with a mid-axillary line of the patient when the medical device holder is applied to the body of the patient.

In another embodiment, a medical device holder for holding and maintaining a position of one or more transducers relative to a body of a patient is provided. The medical device holder includes a base structure configured to be applied to the body of the patient and two or more transducer holders coupled to the base structure, each transducer holder configured to retain a respective transducer therein. The medical device holder also includes a mid-line indicator, wherein two of the two or more transducer holders are arranged symmetrically across the mid-line indicator, and wherein the mid-line indicator is configured to align with a mid-axillary line of the patient when the medical device holder is applied to the body of the patient.

### BRIEF DESCRIPTION OF THE DRAWINGS

FIG. 1 illustrates a conventional transducer that may be held and retained in position by the medical device holders of the present disclosure;

FIG. 2 depicts a first example embodiment of a medical device holder on accordance with the present disclosure, including a belt and a base structure embodied as a plate, the plate having a plurality of transducer holders in an ordered configuration;

FIG. 3 depicts a base structure including a plate that may be used with the medical device holder of FIG. 2;

FIGS. 4 and 5 depict the medical device holder of FIG. 2 in a worn configuration;

FIG. 6 illustrates the base structure of FIG. 3 in a perspective view;

FIGS. 7-9 depict additional embodiments of a base structure that may be used with the medical device holder of FIG. 2;

FIG. 10 depicts a further additional embodiment of a base structure that may be used with the medical device holder of FIG. 2;

FIG. 11 depicts an additional example embodiment of a medical device holder in accordance with the present disclosure, including a base structure embodied as a patch and having a plurality of transducer holders in an ordered configuration;

FIG. 12 depicts another example embodiment of a medical device holder, including a base structure embodied as a patch and having a plurality of transducer holders in a staggered configuration;

FIG. 13 depicts a further example embodiment of a medical device holder, including a base structure embodied as a patch and having a single transducer holder;

FIGS. 14A-14C depict various arrangements of traducer holders relative to a base structure;

FIG. 15 depicts an additional embodiment of a medical device holder in accordance with the present disclosure, including a belt and a base structure embodied as a holder panel;

FIG. 16 depicts the medical device holder of FIG. 15 in a worn configuration;

FIG. 17 depicts an additional embodiment of a medical device holder in accordance with the present disclosure, including a base structure embodied as a patch;

FIG. 18 depicts an example embodiment of a belt that may be used with the medical device holder shown in FIG. 17, including a support plate;

FIG. 19 depicts the medical device holder of FIG. 17 coupled to the belt of FIG. 18, in a worn configuration;

FIG. 20 depicts an additional embodiment of the belt of FIG. 18, including two support plates;

FIG. 21 depicts an additional embodiment of a medical device holder in accordance with the present disclosure, including a belt and one or more base structures embodied as sleeves and having one or more transducer holders;

FIG. 22 is an expanded view of the medical device holder of FIG. 21, depicting the sleeve adjustable relative to the belt; and

FIGS. 23 and 24 depict a further example embodiment of a medical device holder in accordance with the present disclosure, including a belt and a support structure embodied as a retaining clip.

Corresponding reference characters indicate corresponding parts throughout the several views of the drawings. It is understood that that Figures are not necessarily to scale.

### DETAILED DESCRIPTION OF THE DISCLOSURE

The present disclosure relates generally to an apparatus, namely a medical device holder, that includes a base structure (e.g., a plate, patch, panel, etc.) and one or more transducer holders coupled to the base structure. The medical device holder includes indicia identifying the proper placement thereof, relative to the body of a patient, such that the medical device holder maintains the positioning of any transducer(s) to ensure accurate hemodynamic measurements.

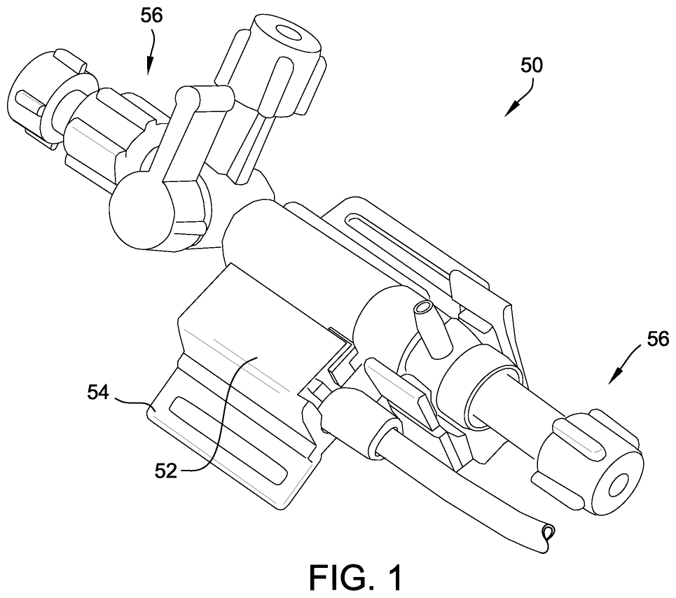

The transducer holders of the present disclosure are suitably shaped and sized as to hold and retain a conventional transducer 50, shown in FIG. 1, although other transducers may be held and retained within the scope of the present disclosure. The transducer 50 includes, broadly, a body 52 (in which the functional elements of the transducer are housed), two lateral wings 54 extending laterally or transversely from the body 52, and coupling extensions 56 that extend longitudinally from the body 52. The coupling extensions 56 are configured to receive various tubing and/or other instruments, as is recognized within the art. Notably, transducers 50 shown in the figures are merely example transducers that may be used with the medical device holders of the present disclosure. Those of skill in the art will appreciate that medical device holder may be modified, as appropriate, to accommodate transducers having any suitable size and/or shape.

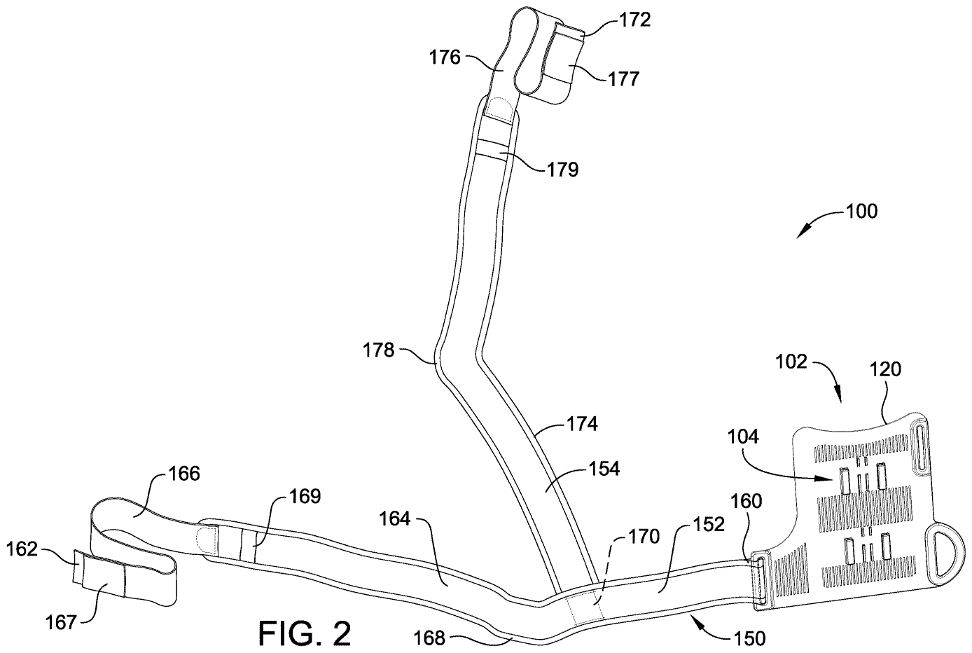

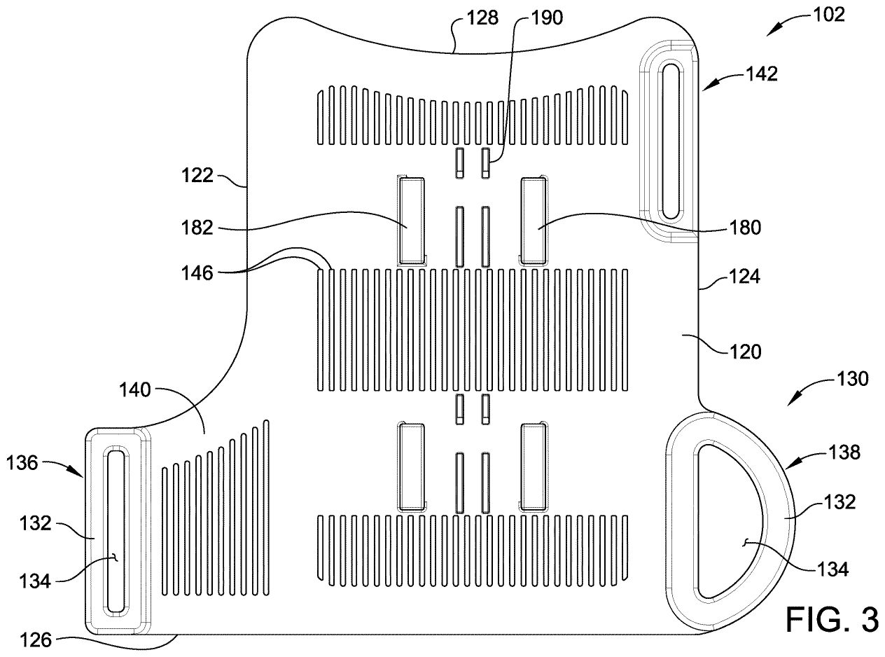

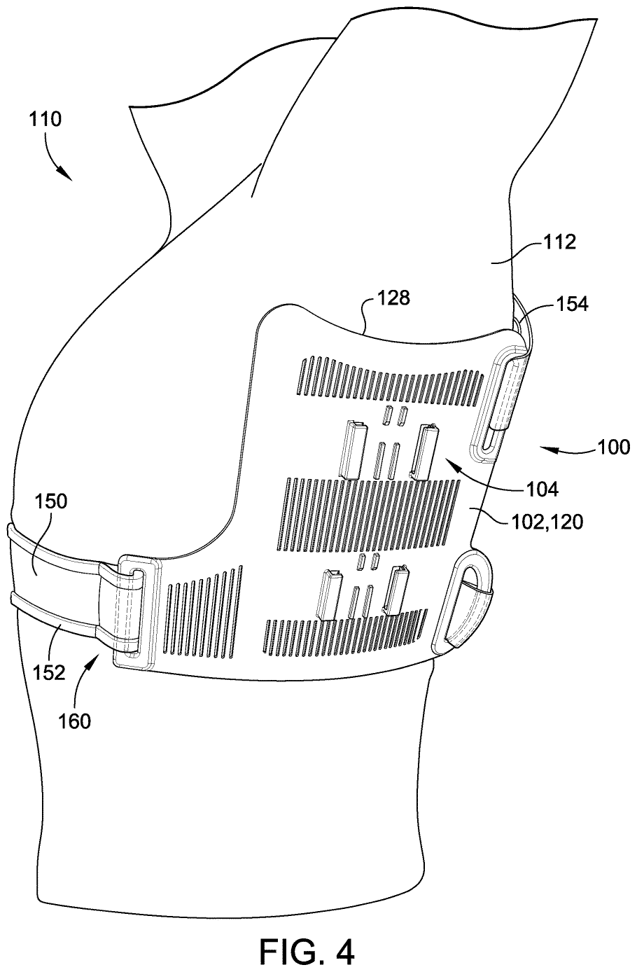

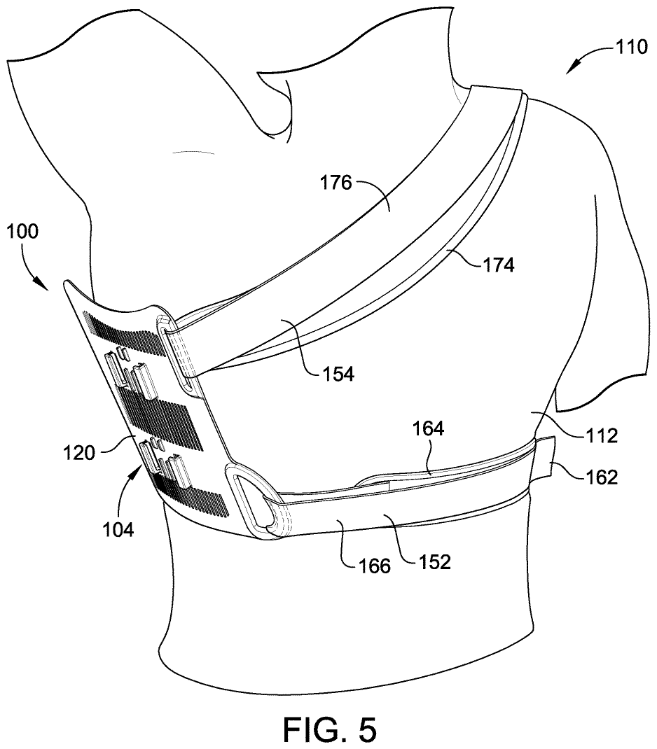

Referring now to the Figures, FIGS. 2-6 depict a first embodiment of a medical device holder 100 in accordance with the present disclosure. In particular, FIG. 2 depicts a plan view of the medical device holder 100, FIG. 3 depicts a base structure 102 of the medical device holder 100 in plan view, FIGS. 4 and 5 illustrate the medical device holder 100 in a worn configuration on a patient 110, and FIG. 6 depicts a perspective view of the base structure 102. The medical device holder 100 includes the base structure 102 and one or more transducer holders 104, which are coupled to and/or integral to the base structure 102. The base structure 102 is configured to be positioned relative to a body 112 of the patient 110 (see FIGS. 4 and 5) for whom hemodynamic monitoring (using transducers 50) is appropriate, such as at a location of the body 112 where the medical device holder 100 is aligned with the mid-axillary line (e.g., at the chest). The medical device holder 100, in the example embodiment, includes a retaining or positioning structure 106, which positions or retains the base structure 102 in the desired position.

A medical device holder 100 with a particular base structure 102 and/or retaining structure 106 used for any patient may be selected based on various parameters, such as a size of the patient, allergies of the patient, patient preference, availability of the medical device holder 100 with a desired base structure 102 and/or retaining structure 106, and the like.

In the embodiment of FIGS. 2-6, the base structure 102 is embodied as a plate 120, and the retaining structure 106 is embodied as a belt 150. As described further herein, the belt 150 includes a primary strap 152 and a stabilizing strap 154.

The plate 120 has a pair of side edges 122, 124 that are oriented parallel to one another. A bottom edge 126 extends between first ends of the side edges 122, 124 and is perpendicular thereto. A top edge 128 extended between second ends (opposite to the first ends) of the side edges 122, 124. In the example embodiment, the top edge 128 is arcuate and inwardly concave; this configuration may improve placement of the medical device holder 100, as well as patient comfort when the medical device holder 100 is worn, as the arcuate top edge 128 accommodates the patient's arm.

In the example embodiment, the plate 120 includes a plurality of primary belt loops 130 to enable coupling the belt 150 to the plate 120. A respective primary belt loop 130 extends laterally outward from each of the side edges 122, 124. Each primary belt loop 130 includes a loop 132 and a cavity 134 through which a respective end or portion of primary strap 152 of the belt 150 is extended. The plate 120 includes two primary belt loops 130, such that the primary strap 152 of the belt 150 may be coupled to each side of the plate 120.

In particular, the plate 120 includes a first primary loop 136 and a second primary loop 138. The first primary loop 136 extends from the first side edge 122 of the plate 120 via an extension arm 140, which is integrally formed with the plate 120. The cavity 134 of the first primary loop 136 corresponds in size to a cross-section of the primary strap 152 of the belt 150. In addition, in the example embodiment, the primary strap 152 is fixedly coupled to the plate 120 via the first primary loop 136. For example, in forming the medical device holder 100, a first end (160, shown in FIG. 2) of the primary strap 152 is fed through the cavity 134, folded back onto the primary strap 152, and fixedly coupled to the primary strap 152, such as by stitching, melting, or an alternative fixation method. The limited size of the cavity 134 (such that relative motion between the belt 150 and the loop 132 is limited) and the integral extension arm 140 enhance the stability and strength of the coupling between the belt 150 and the first primary loop 136.

The second primary loop 138 extends from the second side edge 124 of the plate 120. In the example embodiment, the loop 132 is integrally formed with the plate 120 and extends laterally beyond the second side edge 124 of the plate 120. In such an arrangement, the cavity 134 of the second primary loop 138 is positioned laterally beyond the second side edge 124 of the plate 120. In alternative embodiments, the cavity 134 may be at least partially defined laterally inwardly of the second side edge 124. The cavity 134 of the second primary loop 138 is larger than the cavity 134 of the first primary loop 136 to accommodate a user maneuvering a second end (162, shown in FIG. 2) of the primary strap 152 of the belt 150 through the second primary loop 138, as described further herein. As such, this larger cavity 134 may enhance the ease of use of the medical device holder 100.

Although the belt loops 130 are shown as extending from the side edges 122, 124 adjacent to the bottom edge 126, in some embodiments, the belt loops 130 may extend from the side edges 122, 124 adjacent to the top edge 128 or from a location of each side edge 122, 124 intermediate to the bottom edge 126 and the top edge 128 (e.g., equidistant from the bottom edge 126 and the top edge 128, or closer to the bottom edge 126, or closer to the top edge 128). Moreover, although the belt loops 130 are shown as extending from the side edges 122, 124 opposite to one another (e.g., at the same longitudinal location), in some embodiments, the belt loops 130 may be misaligned or staggered.

In addition, in the example embodiment, the plate 120 includes a stabilizing belt loop 142, which enables coupling of a free end (172, shown in FIG. 2) of the stabilizing strap 154 of the belt 150 to the plate 120. In the example embodiment, the stabilizing belt loop 142 is positioned adjacent to the second side edge 124 of the plate 120, the cavity 134 being inset relative to the second side edge 124. That is, the stabilizing belt loop 142 does not extend past the second side edge 124. In other embodiments, the stabilizing belt loop 142 extends laterally from the second side edge 124 and/or longitudinally (e.g., upwards, with respect to the view of FIG. 3) from the top edge 128.

In some embodiments, the plate 120 is formed with a plurality of slots or apertures 146 therein. The apertures 146 may enhance patient comfort, enabling air flow through the plate 120. The apertures may 146 may affect the rigidity of the plate 120. For example, having more apertures 146 and/or larger apertures 146 (e.g., increasing the overall amount of the surface area of the plate 120 disrupted by apertures 146) may increase flexibility of the plate 120.

The plate 120 may be formed from a rigid material, such that the plate 120 exhibits little to no flexion while applied to the body 112 of the patient 110. Rigidity may be advantageous in ensuring the positioning of the transducer holders 104 does not change once the plate 120 is positioned. The plate 120 may be formed from a flexible material, such that the plate 120 exhibits high flexion while applied to the body 112. Flexibility may be advantageous in improving compliance of the plate 120 to the body 112, which may enhance positioning of the transducer holders 104 and/or enhance comfort of the patient 110. The plate 120 may be formed from a semi-rigid material, such that the plate 120 exhibits an intermediate level of flexion while applied to the body 112. In such instances, the plate 120 may exhibit any or all of the above-described advantages. The plate 120 may be formed from any suitable material, including a hypoallergenic polymeric material, a metal material, or a combination of multiple materials.

The plate 120 may have any suitable size. A length L of the plate 120, measured between the bottom edge 126 and a maximum extent of the top edge 128, may be, for example, between 4 to 12 inches (101.6 to 304.8 millimeters (mm)). A width W of the plate 120, measured between the first side edge 122 and the second side edge 124, may be, for example, between 4 to 10 inches (101.6 to 254 mm).

With reference to FIG. 2, the primary strap 152 of the belt 150 includes a first (fixed) end 160 and a second (free) end 162. The first end 160 is fixedly coupled to the plate 120 as described herein. The free end 162 is free, that is, not fixedly coupled to another component of the medical device holder 100.

In the example embodiment, the primary strap 152 includes a first portion 164 and a second portion 166. The first portion 164 extends from the first end 160 to the second portion 166; the second portion 166 extends from the free end 162 to the first portion 164. The second portion 166 is fixedly coupled to the first portion 164. The second portion 166 includes a fastening component 167 adjacent to the free end 162, for coupling the free end 162 with the first portion 164 (and/or the second portion 166) when the medical device holder 100 is in use, as described further herein. In the example embodiment, the fastening component 167 includes hook-and-loop fasteners. Additionally or alternatively, the fastening component 167 includes any other fastening component (e.g., snap(s), clasp(s), clip(s), buckle(s), etc.), and/or the first portion 164 of the primary strap 152 may include a complementary fastening component (not shown) configured to engage with the fastening component 167 to couple the free end 162 to the first portion 164.

The second portion 166 is formed of a material with high elasticity, and the first portion 164 is formed from a material with low to no elasticity but exhibiting higher comfort characteristics (e.g., softness, padding, etc.). In the example embodiment, the first portion 164 is formed of a material suitable for fastening with hook-and-loop fasteners. Additionally, the first portion 164 includes a strap retainer 169 (e.g., an elastic band) coupled thereto, adjacent to the second portion 166. With this arrangement, the ease of releasably coupling the second end 162 of the primary strap 152 to the plate 120 (as described herein) is enhanced. In other embodiments, the primary strap 152 is formed from a single material (e.g., either the high-elasticity material or the high-comfort material). In still other embodiments, the first portion 164 exhibits some level of elasticity less than that of the second portion 166.

In addition, in the example embodiment, the primary strap 152 is not completely linear or straight, but includes an apex 168 that defines an angular bend in the primary strap 152. The location of apex 168 is selected to improve the arrangement of the primary strap 152 around the body 112 of the patient 110 when the medical device holder 100 is worn. Therefore, although the apex 168 is illustrated as being closer to the first end 160 of the primary strap 152 and within the first portion 164 of the primary strap 152, the apex 168 may be otherwise located in other embodiments (e.g., in the middle of the primary strap 152; closer to the free end 162; within the second portion 166; etc.).

The stabilizing strap 154 includes a first (fixed) end 170 and a second (free) end 172. In the example embodiment, the stabilizing strap 154 is fixedly coupled to the primary strap 152 at the first end 170 thereof. The free end 172 is free, that is, not fixedly coupled to another component of the medical device holder 100. The first end 170 of the stabilizing strap 154 is coupled to the first portion 164 of the primary strap 152, in a location adjacent to the apex 168. In other embodiments, the first end 170 is coupled to the primary strap 152 at any suitable location. The first end 170 may be sewn and/or melted to the primary strap 152. In other embodiments, at least a portion of the stabilizing strap 154 is integrally formed with the primary strap 152.

In the example embodiment, like the primary strap 152, the stabilizing strap 154 includes a first portion 174 and a second portion 176. The first portion 174 extends from the first end 170 to the second portion 176; the second portion 176 extends from the free end 172 to the first portion 174. The second portion 176 is fixedly coupled to the first portion 174. The second portion 176 includes a fastening component 177 adjacent to the free end 172, for coupling the free end 172 with the first portion 174 (and/or the second portion 176) when the medical device holder 100 is in use, as described further herein. In the example embodiment, the fastening component 177 includes hook-and-loop fasteners. Additionally or alternatively, the fastening component 177 includes any other fastening component (e.g., snap(s), clasp(s), clip(s), buckle(s), etc.), and/or the first portion 174 of the stabilizing strap 154 may include a complementary fastening component (not shown) configured to engage with the fastening component 177 to couple the free end 172 to the first portion 174.

The second portion 176 is formed of a material with high elasticity, and the first portion 174 is formed from a material with low to no elasticity but exhibiting higher comfort characteristics (e.g., softness, padding, etc.). In the example embodiment, the first portion 174 is formed of a material suitable for fastening with hook-and-loop fasteners. Additionally, the first portion 174 includes a strap retainer 179 (e.g., an elastic band) coupled thereto, adjacent to the second portion 176. With this arrangement, the ease of releasably coupling the second end 172 of the stabilizing strap 154 to the plate 120 (as described herein) is enhanced. In other embodiments, the stabilizing strap 154 is formed from a single material (e.g., either the high-elasticity material or the high-comfort material). In still other embodiments, the first portion 174 exhibits some level of elasticity less than that of the second portion 176.

In addition, in the example embodiment, the stabilizing strap 154 is not completely linear or straight, but includes an apex 178 that defines an angular bend in the stabilizing strap 154. The location of apex 178 is selected to improve the arrangement of the stabilizing strap 154 around the body 112 of the patient 110 when the medical device holder 100 is worn. Therefore, although the apex 178 is illustrated as being closer to the first end 170 of the stabilizing strap 154 and within the first portion 174 of the stabilizing strap 154, the apex 178 may be otherwise located in other embodiments (e.g., in the middle of the stabilizing strap 154; closer to the free end 172; within the second portion 176; etc.).

In alternative embodiments of the medical device holder 100, the first end 160 of the primary strap 152 may be releasably couplable to the plate 120. For example, the first end 160 and an adjacent region (not shown) of the primary strap 152 may include complementary fastening components (e.g., hook-and-loop fasteners, snap(s), clasp(s), clip(s), buckle(s), etc.). In some alternative embodiments of the medical device holder 100, the stabilizing strap 154 may be releasably couplable to the primary strap 152 via one or more fastening components (e.g., hook-and-loop fasteners, snap(s), clasp(s), clip(s), buckle(s), etc.).

The primary strap 152 and/or the stabilizing strap 154 may have any suitable size, depending on various factors including the size of the patient 110 being monitored. A length L of the primary strap 152 and/or the stabilizing strap 154 therefore may be, for example, between 25 to 65 inches (635 to 1651 mm) to accommodate patients of various sizes. The primary strap 152 and the stabilizing strap 154 may have a same length L, or one strap 152, 154 may be longer than the other of straps 152, 154.

To couple the medical device holder 100 to the patient 110, the plate 120 is engaged against the body 112 of the patient 110 (e.g., against the patient's chest). The primary strap 152 is wrapped around the body 112 of the patient 110, and, with the first end 160 of the primary strap 152 fixedly coupled to the plate 120 and therefore retained in place, the free end 162 of the primary strap 152 is coupled to the plate 120. Specifically, the free end 162 is passed through the second primary loop 138 and is folded back towards the rest the of the primary strap 152. The free end 162 is optionally passed through the strap retainer 169, and is releasably coupled to the first portion 164 of the primary strap 152 (via hook-and-loop engagement between the fastening component 167 and the first portion 164, in the example embodiment). The primary strap 152 is thereby secured around the patient 110.

The position of the plate 120 may be adjusted and/or confirmed. Once the plate 120 is optimally located, the stabilizing strap 154 is wrapped diagonally across the patient (e.g., across the front and then the back of their chest). With the first end 170 of the stabilizing strap 154 fixedly coupled to the primary strap 152 and thereby retained in place, the free end 172 of the stabilizing strap 154 is coupled to the plate. Specifically, the free end 172 is passed through the stabilizing belt loop 142 and is folded back towards the rest the of the stabilizing strap 154. The free end 172 is optionally passed through the strap retainer 179, and is releasably coupled to the first portion 174 of the stabilizing strap 154 (via hook-and-loop engagement between the fastening component 177 and the first portion 174, in the example embodiment). The stabilizing strap 154 is thereby secured around the patient 110, and the plate 120 is secured in its optimal location relative to the patient's body 112.

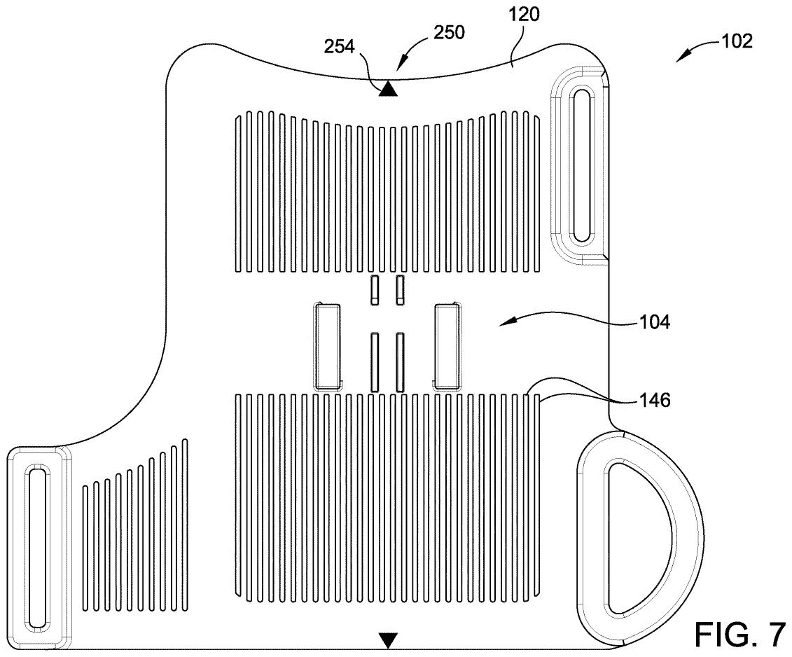

Turning to FIGS. 3 and 6-9, the base structure 102 (e.g., the plate 120) has a plurality of transducer holders 104 coupled thereto. The transducer holders 104 are arranged on an outward facing surface of the base structure 102. In the example embodiment, the transducer holders 104 are integrally formed (e.g., molded, printed, etc.) with the base structure 102. In other embodiments, the transducer holders 104 are separate components coupled to the base structure 102, such as in the embodiment of FIG. 10 (described below).

Each transducer holder 104 includes a pair of wing retainers 180 configured to receive and retain the lateral wings 54 of the transducer 50 (as shown in FIG. 1). Each wing retainer 180 includes a front panel 182, a side panel 184, and a bottom panel 186. The side panel 184 and the bottom panel 186 each extend outwardly from the base structure 102, oriented perpendicular to the base structure 102. The front panel 182, oriented parallel to the base structure 102, is coupled to (e.g., integrally formed with) the side and bottom panels 184, 186. The panels 182, 184, 186 collectively define a slot or gap 188 (see FIG. 6) configured to receive the respective lateral wing 54. The lateral wing 54 rests against the bottom panel 186 to maintain the lateral wing 54 within the gap 188. The particular length and width of the wing retainers 180 as well as the dimensions (e.g., “depth”) of the gap 188 are selected such that the wing retainers 180 have a suitably tight engagement with the lateral wings 54, such that the lateral wings 54 are not dislodged from the wing retainers 180 when the medical device holder 100 is applied to and/or worn by the patient 110.

In addition, each transducer holder 104 includes a set of ridges 190 configured to orient the transducer 50 within the transducer holder 104 and relative to the base structure 102.

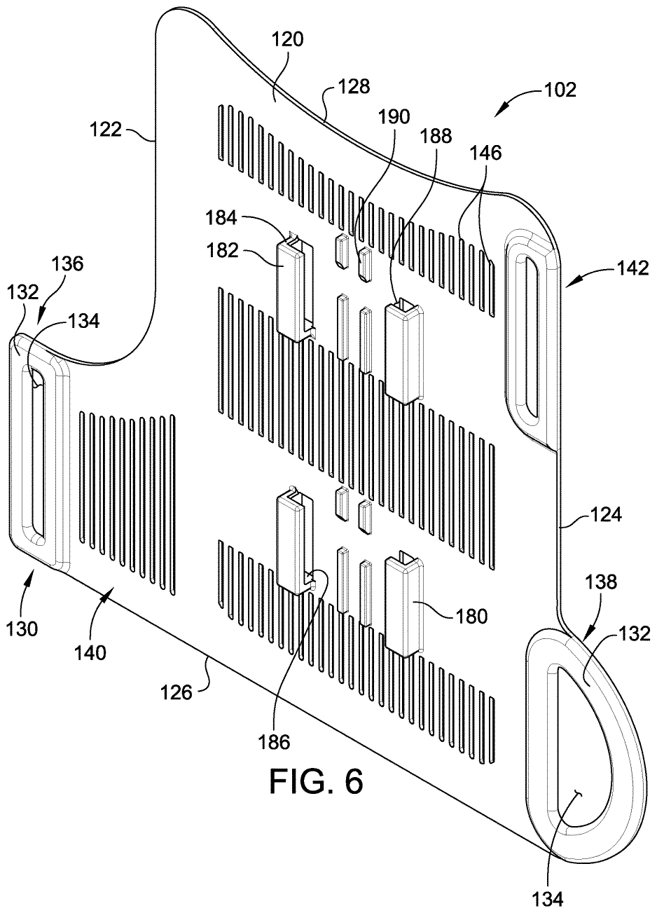

FIGS. 6-9 depict base structures 102 (e.g., plates 120) that may be used with the medical device holder 100, having various configurations of transducer holders 104. In particular, FIG. 6 depicts two transducer holders 104 in an aligned longitudinal configuration. Notably, two or more transducer holders 104 may be arranged in the aligned longitudinal configuration. FIG. 7 depicts a single transducer holder 104 in a centered configuration. For instance, the transducer holder 104 is centered laterally and longitudinally with respect to the plate 120. In other embodiments, a single transducer holder 104 may not be longitudinally centered (e.g., may be closer to the bottom edge 126 or the top edge 128) but is optimally laterally centered. FIG. 8 depicts the transducer holders 104 arranged in a staggered configuration. In the staggered configuration, two transducer holders 104 are laterally aligned with one another in a first row, and one or more other transducer holders 104 are arranged in another, longitudinally offset row (which may be on either side of the first row). FIG. 9 depicts the transducer holders 104 arranged in a patterned configuration. In the patterned configuration, two transducer holders 104 are laterally aligned with one another in a first row and two transducer holders 104 are laterally aligned with one another in a second row.

As shown in FIG. 10, in some embodiments, the base structure 102 does not include any transducer holders. Instead, the base structure 102, embodied as a plate 120′, includes one or more windows 198 defined therein. Each window 198 is configured to receive a respective further base structure therein, such a base structure embodied as a patch (described further herein). Each patch may be coupled to an outward-or inward-facing surface of the plate 120′, exposing the transducer holders 104 through the window 198. In some embodiments, the patch retaining the transducer holders 104 may be coupled to the skin of the patient 110, and the plate 120′ facilitates retaining the patch and the transducer holders 104 in place.

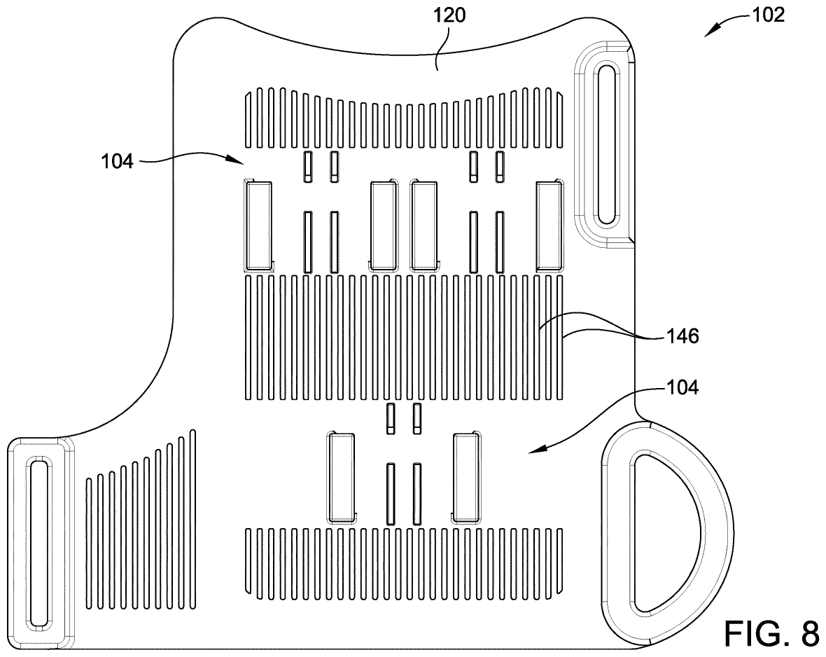

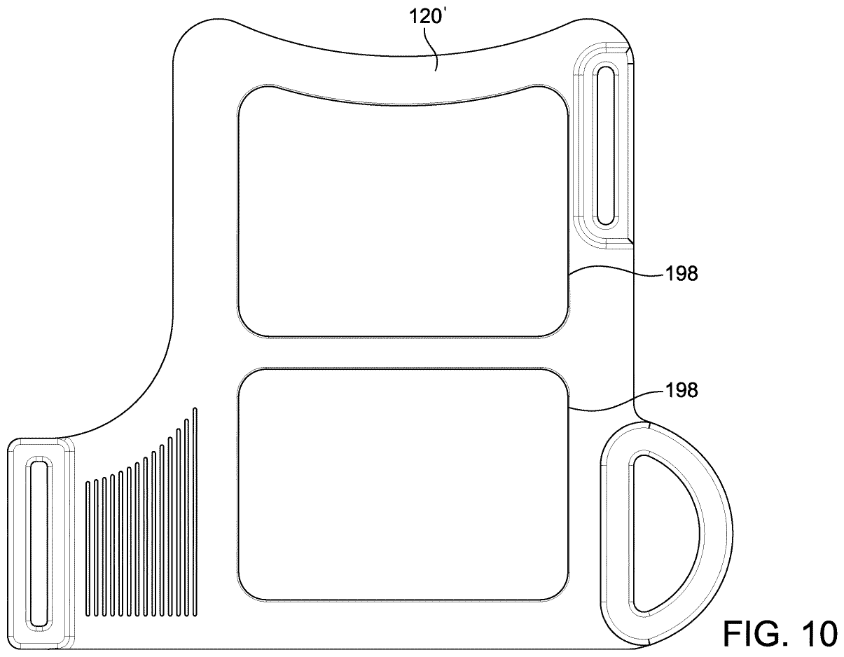

FIGS. 11-13 depict additional embodiments of a medical device holder 200, in which the base structure 102 is embodied as a patch 220. The medical device holder 200 includes one or more transducer holders 204 coupled to the base structure 102/220.

More specifically, the base structure 102 includes a patch 220 configured to be applied to the body of a patient (e.g., the patient 110), specifically the skin of the patient. The patch 220 includes a body-facing bottom surface 222 and an opposing top surface 224. In the example embodiment, the patch 220 is an adhesive patch and includes adhesive (not shown) applied to the body-facing surface 222 as well as one or more protective covers (also not shown) that overlay the adhesive until the patch 220 is used. The patch 220 is therefore easily applied when intended for use in, for example, a medical setting, in that a user (e.g., a medical professional) need only remove the protective cover(s) to ready the patch 220 for application to the body of the patient. In at least some embodiments, the patch 220 is a transparent patch, such that the body of the patient is at least partially visible through the patch 220. The patch 220 may be formed from any suitable material, in particular, a flexible and elastic material such that the patch 220 may flex with movement of the patient. For example, the patch 220 may be formed from a polymeric sheet material (e.g., polyester, nylon), a woven fabric material, or any other suitable material.

The patch 220 may have any suitable size, depending on the number of transducers 50 intended to be held by the patch 220. A length L of the patch 220 may be, for example, between 2 to 6 inches (50.8 to 152.4 millimeters (mm)) to accommodate a single transducer 50 and transducer holder 104; between 2 to 8 inches (50.8 to 203.2 mm) to accommodate two transducers 50 and transducer holders 104 in any longitudinal arrangement; or between 2 to 12 inches (50.8 to 304.8 mm) to accommodate three transducers 50 and transducer holders 104 in any longitudinal arrangement. A width W of the patch 220 may be, for example, between 1 to 6 inches (25.4 to 152.4 mm) to accommodate a single transducer 50 and transducer holder 104, or between 1 to 10 inches (25.4 to 254 mm) to accommodate two transducers 50 and transducer holders 104 in any lateral arrangement.

The transducer holders 104 are arranged on an outward facing surface of the respective base structure 102. For example, the transducer holders 104 are arranged on the top surface 224 of the patch 220. Each transducer holder 104 includes a pair of wing retainers 230 and a strap 232. The wing retainers 230 are configured to receive and retain the lateral wings 54 of the transducer 50 (as shown in FIG. 1), and the strap 232 is configured to receive and retain the body 52 (also shown in FIG. 1) of the transducer 50.

Each wing retainer 230 has a panel 234 that extends between two ends 236. The ends 236 are coupled to the base structure 102, such as by sutures, adhesive, bonding, or any other suitable coupling method. The panel 234, between the two ends 236, remains decoupled from the base structure 102, such that a gap (not shown) is defined between the panel 234 and the base structure 102. The wing retainers 230 are configured to hold the lateral wings 54 (or other laterally extending elements) of the transducer 50, when the transducer 50 is inserted into the transducer holder 104. Specifically, the lateral wings 54 are inserted into the gaps between the panels 234 and the base structure 102. The particular length and width of the wing retainers 230 as well as the dimensions (e.g., “depth”) of the gap are selected such that the wing retainers 230 have a suitably tight engagement with the lateral wings 54, such that the lateral wings 54 are not dislodged from the wing retainers 230 when the medical device holder 200 is applied to and/or worn by the patient 110.

In some embodiments, the wing retainers 230 are formed from a suitable material, which may be a flexible, elastic material that enables the wing retainers 230 to stretch around the wings 54 of the transducer 50. For example, the wing retainers 230 may be formed from a polymeric sheet material, a woven fabric material, an elastic (e.g., rubber) material, or any other suitable material. In some embodiments, where the base structure 102 is the patch 220, the wing retainers 230 are formed separately from the patch 220 and are coupled to the patch 220 during manufacturing of the medical device holder 200. In other embodiments where the base structure 102 is the patch 220, the wing retainers 230 are formed from the patch material. For example, the material of the patch 220 is cut to form edges of the panel 234, and the panel 234 is raised to form the gap into which the transducer lateral wings 54 are inserted.

The strap 232 of the transducer holder 104 has two ends 238 at which the strap 232 is coupled to the base structure 102. A gap (not shown) is defined between the strap 232 and the base structure 102. Alternatively, the strap 232 forms a continuous circle (or other shape) and lacks any ends. In such cases, one circumferential portion of the strap 232 is coupled to the base structure 102. The strap 232 is coupled to the base structure 102 by sutures, adhesives, bonding, or any other suitable coupling method. The strap 232 is configured to hold the body 52 of the transducer 50, when the transducer 50 is inserted into the transducer holder 104. Specifically, the body 52 is inserted through the gap between the strap 232 and the patch 220. The particular length of the strap 232 is selected such that the strap 232 has a suitably tight engagement with the body 52, such that the body 52 is not dislodged from the strap 232 when the medical device holder 200 is applied to and/or worn by the patient. In some embodiments, the strap 232 is formed from an elastic material that enables the strap 232 to stretch around the body 52 of the transducer 50. The strap 232 may be formed from any other suitable material.

In some embodiments, the transducer holders 104 further include a secondary fastener 240. The secondary fastener 240 is configured to retain the body 52 and/or a coupling extension 56 (as shown in FIG. 1) of the transducer 50. In the illustrated embodiment, the secondary fastener 240 includes a belt-like strap 242, which extends between a first end 244 and a second end 246. The belt-like strap 242 is coupled to the base structure 102 at a location intermediate the first and second ends 244, 246, such as by sutures, adhesives, bonding, or any other suitable coupling method. The first end 244 includes a head or other suitable receiving element configured to receive the second end 246, which may include a tail. The secondary fastener 240 is configured to hold the body 52 and/or coupling extension 56 of the transducer 50, when the transducer 50 is inserted into the transducer holder 104. Specifically, the transducer 50 is inserted into the strap 232 and the wing retainers 230, as described above. The secondary fastener 240 is then engaged with the transducer 50 to further prevent movement of the transducer 50 when the medical device holder 200, 300 is applied to and/or worn by the patient. In the example embodiment, the first and second ends 244, 246 of the belt-like strap 242 are drawn together, the second end 246 is received in the first end 244, and the belt-like strap 242 is tightened and fixed in a tightened configuration (e.g., similar to a “cable tie”). The secondary fastener 240 may alternatively be any other suitable fastening element, such as a duplicate of the strap 232.

Those of skill in the art will appreciate that, in various embodiments, a given transducer holder 104 may include any suitable combination of wing retainers 230, straps 232, and/or secondary fasteners 240. For example, a transducer holder 104 may include straps 232 but not include wing retainers 230 or secondary fasteners 240 (e.g., resulting in reduced manufacturing costs). In another example, a transducer holder 104 may include straps 232 and wing retainers 230, but not secondary fasteners 240.

The medical device holders of the present disclosure (e.g., the medical device holder 100, 200, and/or any other medical device holder or component thereof that is described herein) also includes a mid-line indicator 250, which is configured to align with a mid-axillary line of the patient (e.g., the patient 110) when the medical device holder 100, 200 is applied to the patient 110, thereby enabling the transducer holders 104 (and, thereby, the transducers 50 retained therein) to be accurately and precisely located with respect to the body 112 of the patient 110. Specifically, as described herein, the transducer 50 should be placed at the appropriate mid-axillary level to ensure accurate leveling (zeroing) and pressure measurements. In general, the most optimal way to ensure the transducer 50 is so located is to place it directly onto the surface through which the mid-axillary level is defined. The mid-line indicator 250 being part of the medical device holder 100, 200 facilitates intuitive, rapid, and accurate placement of the medical device holder 100, 200 which then maintains placement of the transducers 50 at the accurate level.

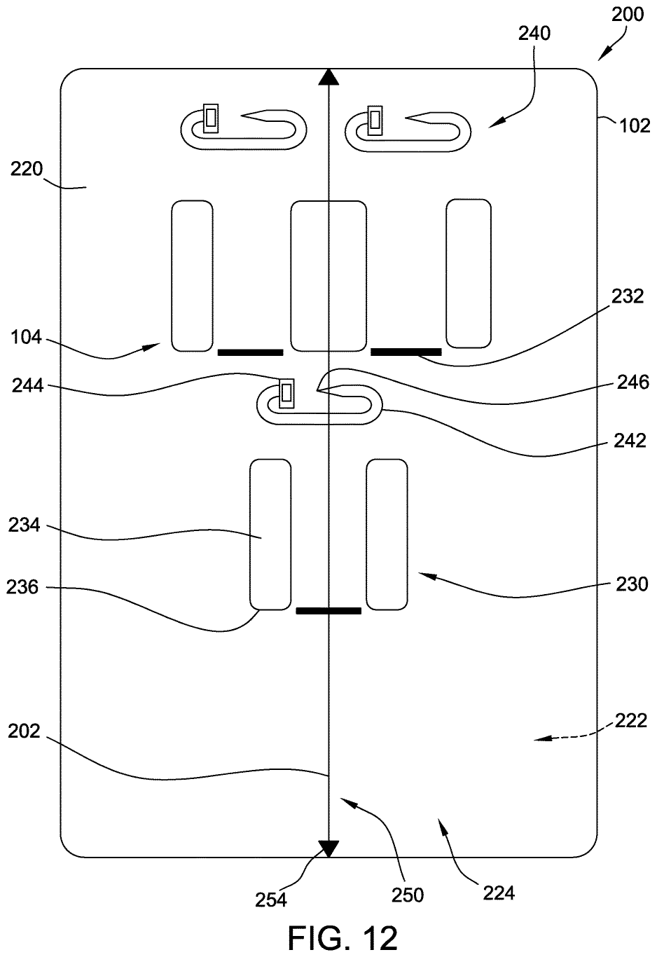

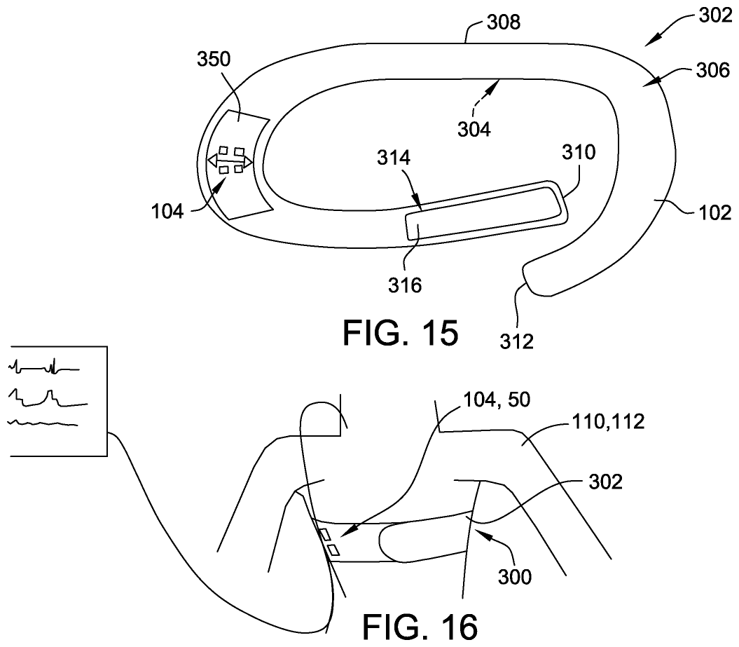

In the example embodiment, the mid-line indicator 250 is a straight line 252 bounded by two end icons 254, depicted as arrowheads. The line 252 may be continuous or discontinuous (e.g., dashed). In some embodiments (e.g., as shown in FIG. 7), the mid-line indicator 250 does not include the straight line 252 but includes the end icons 254 (e.g., arrowheads) that direct placement of the medical device holder, as described herein. The end icons 154 may accordingly be located adjacent opposite side edges of the corresponding base structure. The mid-line indicator 250 is printed on the plate 120, the patch 220, or a holder panel (e.g., a holder panel 350, shown in FIGS. 15-22) on which the transducer holders 104 are arranged. More specifically, the mid-line indicator 250 is positioned relative to the location of the transducer holders 104. For example, in the example embodiment, the mid-line indicator 250 is selectively positioned relative to the base structure 102 to pass through a longitudinal midline of at least one transducer holder 104 (and, thereby, a longitudinal midline of at least one transducer 50). The mid-line indicator 250 is provided to enable a medical professional to visualize the relative placement of the transducer 50 (retained by the medical device holder 100) with the mid-axillary level, by visually aligning the mid-line indicator 250 with the mid-axillary level of the body 112 of the patient 110. Accordingly, when the mid-line indicator 250 is accurately placed relative to the mid-axillary level of the body 112 of the patient 110, the medical professional may apply the medical device holder 100 and be assured that any transducers 50 held by the medical device holder 100, 200 are accurately placed.

In some embodiments, such as where the base structure 102 is a plate 120 or a patch 220, the mid-line indicator 250 is provided directly on the top or outer-facing surface of the base structure 102. In the example embodiment, the mid-line indicator 250 extends down a longitudinal mid-line of the plate 120/patch 220 and bisects the plate 120/patch 220, further increasing the intuitive placement of the medical device holder 100, 200. In alternative embodiments, the mid-line indicator 250 may be other than centrally located on the plate 120/patch 220, but is still positioned to bisect at least one transducer holder 104. Additionally, the mid-line indicator 250 extends from one end of the plate 120/patch 220 to the other end of the plate 120/patch 220, the end icons 254 of the mid-line indicator 250 abutting respective edges of the plate 120/patch 220. This positioning further increases the ease of use of the medical device holder 100, 200, because the mid-line indicator 250 is optimally visible during placement of the medical device holder 100, 200.

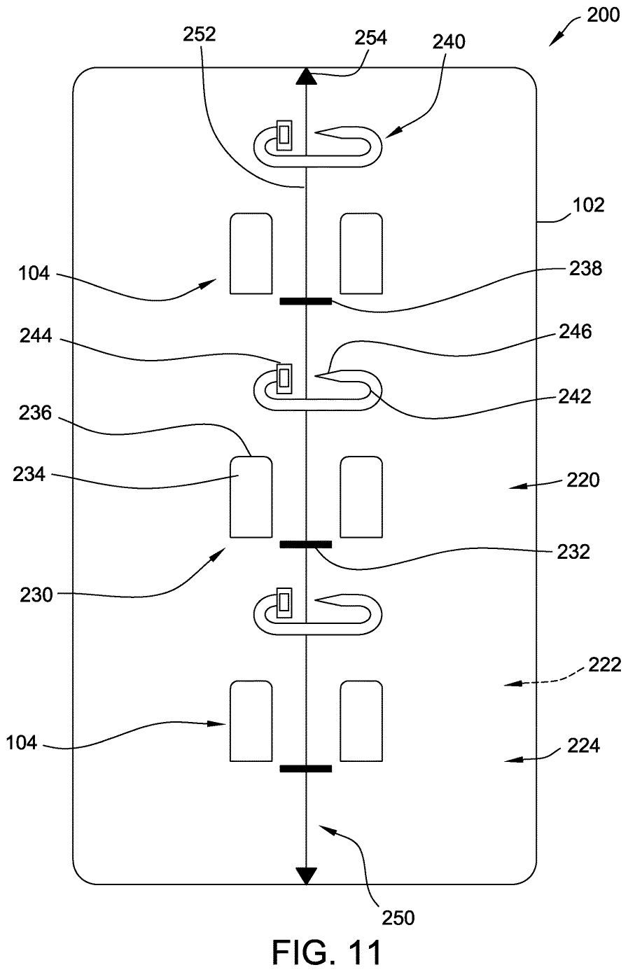

FIGS. 14A-14C illustrate various configurations of transducer holders 104 that may be implemented with the medical device holders of the present disclosure. The transducer holders 104, as shown, may be coupled to a base structure 102, which may be embodied as a plate (e.g., as in FIGS. 2-9), a patch (e.g., as in FIGS. 11-13), or a belt (e.g., as in FIGS. 15-22). FIG. 14A depicts the transducer holders 104 arranged in an aligned longitudinal configuration (as also shown in FIG. 11). Two or more transducer holders 104 may be arranged in the aligned longitudinal configuration. This longitudinally aligned configuration would ensure all transducers 50 held by the transducer holders 104 would be maintained in alignment with the mid-axillary line (when the medical device holder 100, 200 is accurately placed on the body 112 of the patient 110).

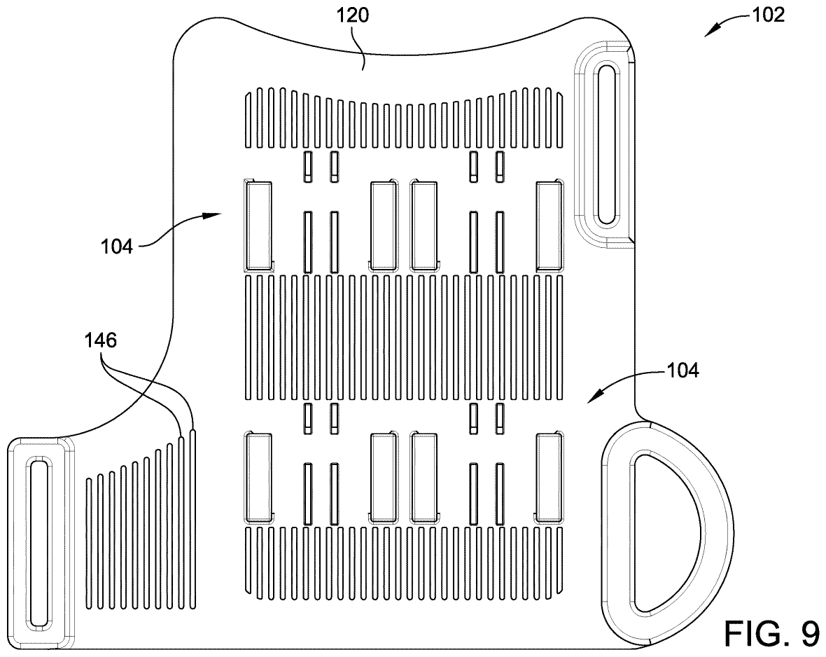

FIG. 14B depicts the transducer holders 104 arranged in a staggered configuration (as also shown in FIG. 12). In the staggered configuration, two transducer holders 104 are laterally aligned with one another in a first row, and one or more other transducer holders 104 are arranged in another, longitudinally offset row (which may be on either side of the first row). When transducer holders 104 are arranged in a staggered configuration, the laterally aligned transducers holders 104 are positioned symmetrically across the mid-line indicator 250, and the longitudinally offset transducer holder 104 is positioned such that the mid-line indicator 250 bisects the transducer holder 104.

FIG. 14C depicts the transducer holders 104 arranged in a patterned configuration. In the patterned configuration, two transducer holders 104 are laterally aligned with one another in a first row and two transducer holders 104 are laterally aligned with one another in a second row. In this patterned configuration, the transducer holders 104 in each respective row are positioned symmetrically across the mid-line indicator 250, and no transducer holder 104 is bisected by the mid-line indicator 250 (but the mid-line indicator 250 preferably bisects the base structure 102 on which the transducer holders 104 are arranged).

FIGS. 15-22 illustrate another embodiment of a medical device holder 300, in which the transducer holders 104 are coupled to a holder panel 350.

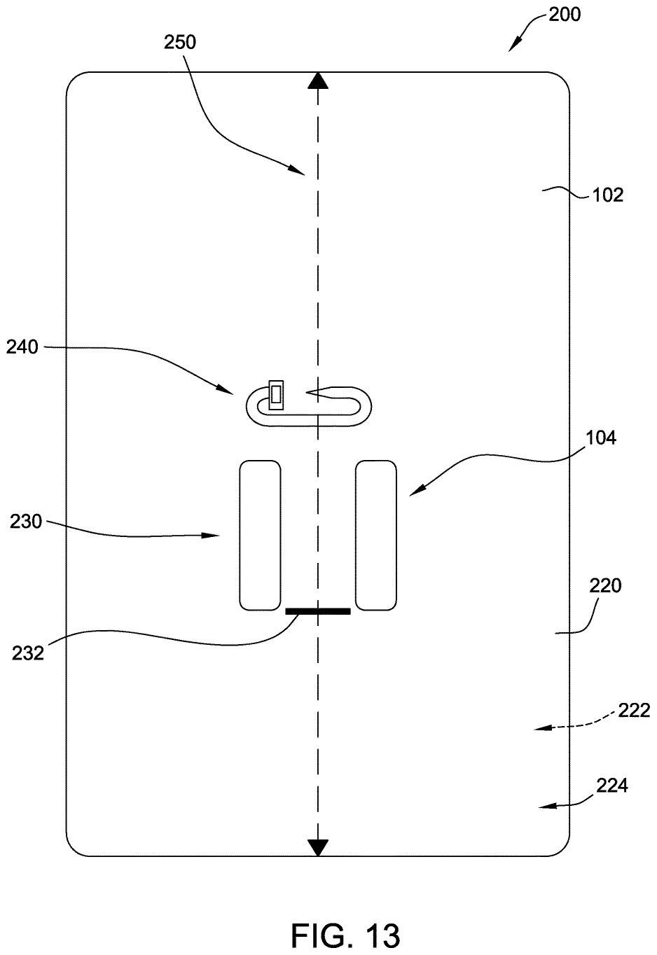

In FIGS. 15 and 16, the medical device holder 300 includes a base structure 102 embodied as a belt 302 that is configured to be wrapped and secured around the body of a patient (e.g., the patient 110). The belt 302 includes a body-facing inner surface 304 and an opposing outer surface 306. A strap 308 of the belt 302 extends between a first end 310 and a second end 312 of the belt 302. The strap 308 is formed from a material that ensures comfort for the wearer, such as a woven or non-woven fabric material, which may exhibit high or low elasticity. The belt 302 is secured in place relative to the body 112 of the patient 110 by one or more fasteners 314. In the illustrated embodiment, the fasteners 314 include opposing strips 316 of hook-and-loop fasteners, where one strip 316 is coupled to the inner surface 304 of the strap 308 (e.g., adjacent one of the first end 310 and the second end 312) and one strip 316 is coupled to the outer surface 306 of the strap 308 (e.g., adjacent the other of the first end 310 and the second end 312). To secure the belt 302, the belt 302 is appropriately tightened around the body 112 of the patient 110, and the strips 316 are engaged with one another. In other embodiments, the fasteners 314 may additionally or alternatively include buttons, snaps, zippers, combinations thereof, and/or any other suitable fastening component(s). In the example embodiment, the belt 302 also includes one more supplemental fasteners (not shown) that are configured to improve engagement and coupling between the belt 302 and the body 112 of the patient 110. The supplemental fasteners may include, for example, adhesive patches coupled to the inner surface 304 of the belt and applicable to the skin of the patient 110.

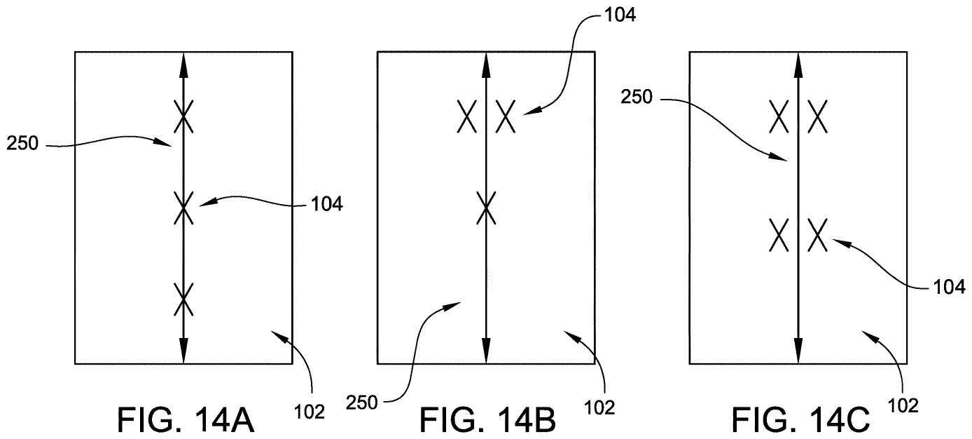

In another embodiment, as shown in FIGS. 17-20, a medical device holder 300 also includes a belt 302. The medical device holder 300 also includes an adjustable assembly 352 coupled to the belt 302 and configured to retain a holder panel 350. In this embodiment, the holder panel 350 is a disposable or single-use patch including one or more transducer holders 104. The holder panel 350 may be distinguished from the patch 220 in that the patch 220 is designed and configured to be applied directly to the patient 110, whereas the holder panel 350 is designed and configured to be applied to a base structure 102 (e.g., a belt) that is then applied to the patient 110.

In particular, the adjustable assembly 352 includes a support surface 354 configured to receive the holder panel 350. The support surface 354 may be a planar surface having a size and/or shape similar to a size and/or shape of the holder panel 350. Specifically, providing the support surface 354 with limited variability for placement of the holder panel 350 relative thereto may improve the reliability of the medical device holder 300 in use, when coupled to the belt 302 via the adjustable assembly 352. In the example embodiment, the holder panel 350 includes an adhesive backing (not shown) that facilitates attaching the holder panel 350 to the support surface 354 of the adjustable assembly 352. In other embodiments, a separate adhesive or other attachment feature (e.g., one or more clips) may be used to couple the holder panel 350 to the support surface 354.

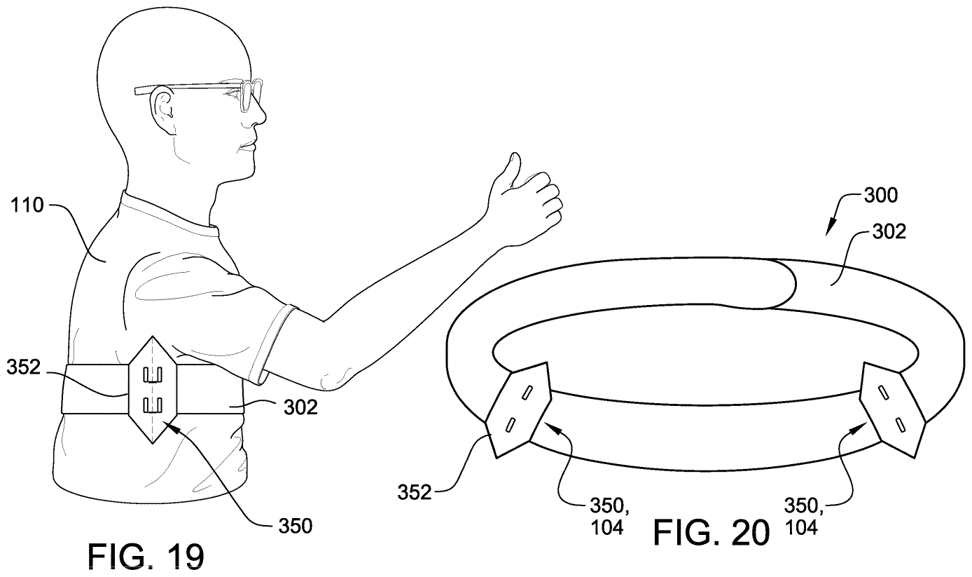

The medical device holder 300 may include one or more adjustable assemblies 352. For example, as shown in FIG. 20, two adjustable assemblies 352 may be positioned on opposite sides of the belt 302 to facilitate mid-axillary alignment. Alternatively, the medical device holder 300 may include any suitable number and arrangement of adjustable assemblies 352 (e.g., one adjustable assembly 352 on a first side of the belt 302 and two adjustable assemblies 352 on a second side of the belt 302, or two adjustable assemblies 352 on each side of the belt 302).

In some embodiments, the adjustable assemblies 352 are coupled to the belt 302 by a sleeve component (not shown) of the adjustable assembly 352 through which the belt 302 is passed. The adjustable assembly 352 can then be translated (e.g., slid) relative to the belt 302. In some embodiments, the adjustable assembly 352 includes a fastener (not shown) coupled to an inward-facing surface thereof, such as a hook-and-loop fastener, snap(s), clip(s), pin(s), etc. The fastener(s) may be used to releasably couple the adjustable assembly 352 to any desired location along the belt 302. It should be understood that, when the holder panel 350 is coupled to the adjustable assembly 352, changing the position of the adjustable assembly 352 changes the position of the transducer holder(s) 104 on the holder panel 350, relative to the base structure 102 (e.g., the belt 302).

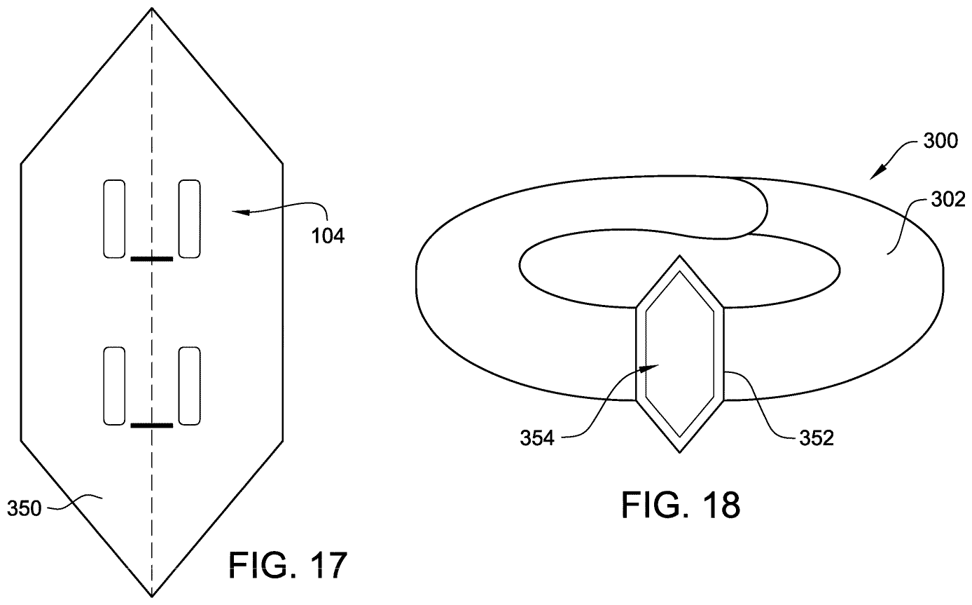

In operation, the belt 302 may be wrapped around the patient 110 (e.g., as shown in FIG. 19), and the holder panel 350 may be coupled to the adjustable assembly 352. Alternatively, the holder panel 350 may be coupled to the adjustable assembly 352 before the belt 302 is arranged around the patient 110. The adjustable assembly 352 facilitates adjusting the location of the holder panel 350—and, thereby, the transducer holder(s) 104 and any transducer(s) 50 held thereby—relative to the mid-axillary line of the patient 110, as described elsewhere herein.

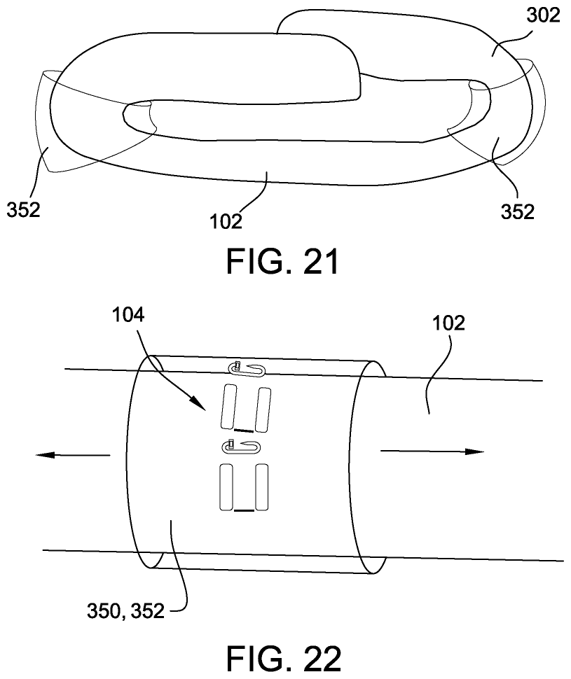

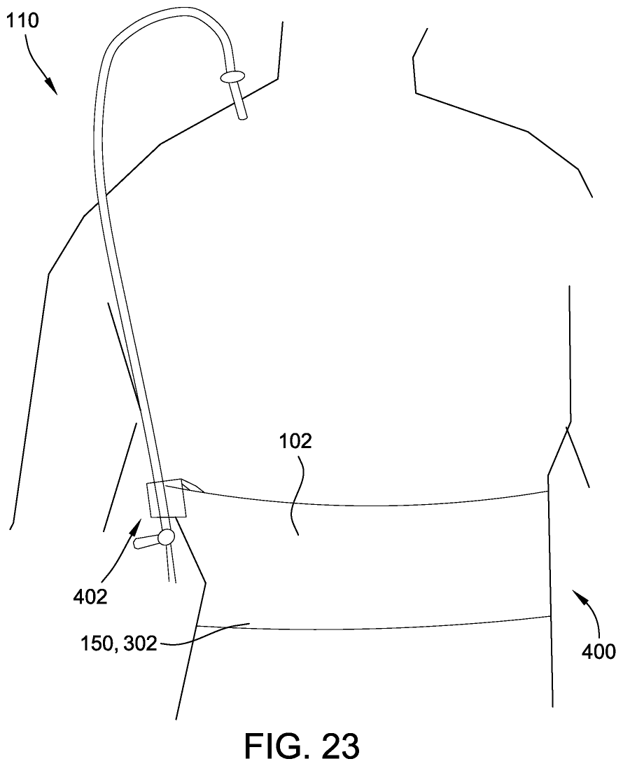

FIGS. 21 and 22 depict another embodiment of the medical device holder 300, including the belt 302 and the adjustable assemblies 352. In this embodiment, the adjustable assemblies 352 are embodied as sleeve-like components. In this embodiment, the belt 302 includes two adjustable assemblies 352 coupled thereto. The adjustable assemblies 352 may be positioned on opposite sides of the belt 302 to facilitate mid-axillary alignment. Alternatively, the belt 302 may include any suitable number and arrangement of adjustable assemblies 352 (e.g., one adjustable assembly 352 on a first side of the belt 302 and two adjustable assemblies 352 on a second side of the belt 302, or two adjustable assemblies 352 on each side of the belt 302). The base structure 102 is threaded through the adjustable assemblies 352 such that the adjustable assemblies 352 may be slid along the base structure 102 to be positioned at a desired location. Further, the base structure 102 and/or the adjustable assemblies 352 may include one or more engagement features (e.g., adhesive, hook and loop fasteners, etc.) for securing the position of the adjustable assemblies 352 relative to the base structure 102 at a particular location.

As shown in FIG. 22, each adjustable assembly 352 includes one or more transducer holders 104 and therefore may itself be considered a holder panel 350. Changing the position of the adjustable assemblies 352 changes the position of the transducer holders 104 relative to the base structure 102. The adjustable assemblies 352 may be made of any suitable material.

In various embodiments of the present disclosure, the belt (e.g., the belt 150 and/or the belt 302) may be constructed from a sterilizable material, such that the belt may be reused. Disposable or single-use patches (e.g., patches 220) and/or holder panels (e.g., holder panels 350), however, may be advantageous as providing a more sterile medical device holder, in at least some instances. An adjustable assembly (e.g., the adjustable assembly 352) may be decouplable from the belt such that the adjustable assembly may be sterilized for reuse. Alternatively, the adjustable assembly may be a single-use component.

In some embodiments, the transducer holders 104 are coupled directly to the outward-facing surface of the base structure 102, as shown in the embodiments of FIGS. 2-9 (e.g., where the base structure 102 is a plate) or FIGS. 11-13 (e.g., where the base structure 102 is a patch). In other embodiments, each transducer holder 104 (or combinations thereof) may be formed as part of a separate component, such as the holder panel 350, such as in the embodiment of FIG. 10 (where the base structure is a windowed plate), or FIGS. 15-22 (where the base structure 102 is a belt), and the holder panel 350 is coupled to the base structure 102. The holder panel 350 can be coupled to any suitable base structure 102, including a plate 120, a patch 220, or a belt 302 (and/or an adjustable assembly 352). This arrangement may have the advantage of manufacturing efficiency, as holder panels 350 can be formed in preset configurations and later applied to a base structure 102.

In some embodiments, where the transducer holders 104 are arranged on a holder panel 350 before being coupled to a base structure 102, the mid-line indicator 250 is provided directly on a top surface (not specifically shown) of the holder panel 350. In the example embodiment, the mid-line indicator 250 extends down a longitudinal mid-line of the holder panel 350 and bisects the holder panel 350. In alternative embodiments, the mid-line indicator 250 may be other than centrally located on the holder panel 350, but is still positioned to bisect at least one transducer holder 104. Additionally, the mid-line indicator 250 extends from one end of the holder panel 350 to the other end of the holder panel 350, the end icons 254 (e.g., arrowheads) of the mid-line indicator 250 abutting respective edges of the holder panel 350. The holder panel 350 is coupled to the base structure 102 in a position that ensures visibility of the mid-line indicator 250.

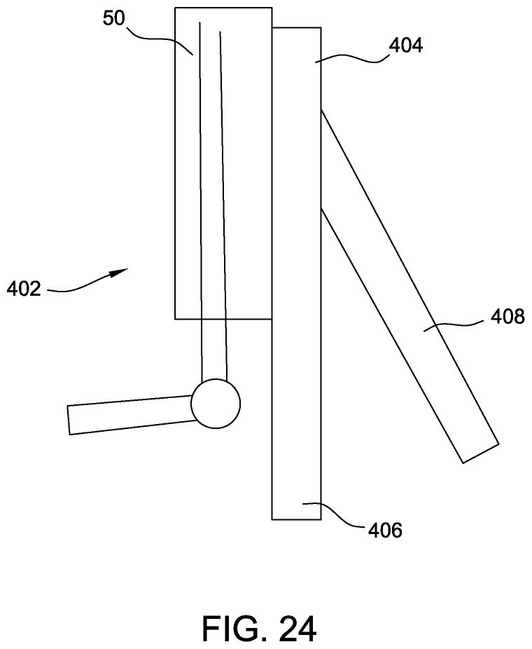

FIGS. 23 and 24 depict another alternative embodiment of a medical device holder 400 including a clip assembly 402 that may be used with a base structure 102 such as a belt (e.g., the belt 150 or the belt 302). As shown in FIG. 24, the clip assembly 402 includes a transducer 50 coupled to a clip 404 (e.g., using one or more adhesive, fasteners, etc.). The clip 404 includes a first arm 406 and a second arm 408 that are biased towards one another (e.g., using a spring).

As shown in FIG. 23, the clip 404 is configured to engage the base structure 102 (i.e., by clamping the base structure 102 between the first and second arms 406 and 408). To change a position of the transducer 50 relative to the base structure 102 and the patient 110, a user may disengage the clip 404 from the base structure 102, move the clip assembly 402 to a desired location, and reengage the clip 404 with the base structure 102.

Those of skill in the art will appreciate the clip 404 is an example mechanism for securing the transducer 50 to a base structure 102 such as a belt. Other suitable mechanisms (e.g., adhesive, hook-and-loop fasteners, etc.) may be additionally or alternatively used.

It should be understood that disclosure related to various components herein may apply equally to other like components. For instance, description of the belt 150 may apply to the belt 302 and vice versa.

The medical device holders of the present disclosure provide advantages over other solutions for holding transducers during hemodynamic monitoring procedures. For instance, the transducer holders of the present disclosure may hold and retain conventional transducers, and may not need to be “redesigned” or otherwise manipulated to retain other types of medical transducers. Moreover, the exchange of transducers is simple and straightforward with the transducer holders of the present disclosure. These benefits are further realized with the medical device holders including the mid-line indicators to facilitate simple, straightforward, and accurate placement of the transducers for accurate measurements. Still further, the transducers are maintained in these preferred positions even when the patient changes position, because the transducer accommodate patient movement (even if the patient is sitting upright or moving). Additionally, these benefits may be realized with different combinations of features of the medical device holders, adjustable assemblies, and/or medical device holder retainers described herein. These benefits also may be realized by implementing the medical device holders of the present disclosure in other monitoring environments, such as in hemodynamic exercise studies, where the transducer holders would maintain positioning of the transducer even during patient exercise, improving the accuracy of pressure measurements.

While embodiments of the present disclosure have been described, it should be understood that various changes, adaptations and modifications may be made therein without departing from the spirit of the disclosure and the scope of the appended claims. Further, all directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. It is intended that all matter contained in the above description or shown in the accompanying drawings shall be interpreted as illustrative only and not limiting. Changes in detail or structure may be made without departing from the spirit of the disclosure as defined in the appended claims.

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments described and that modifications and other embodiments are intended to be included within the scope of the appended claims. Although specific terms are employed herein, they are used in a generic and descriptive sense only and not for purposes of limitation.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A medical device holder for holding and maintaining a position of one or more transducers relative to a body of a patient, the medical device holder comprising:
a base structure configured to be applied to the body of the patient;
one or more transducer holders coupled to the base structure, each transducer holder configured to retain a respective transducer therein; and
a mid-line indicator that bisects at least one of the one or more transducer holders, wherein the mid-line indicator is configured to align with a mid-axillary line of the patient when the medical device holder is applied to the body of the patient.

2. The medical device holder of claim 1, wherein the mid-line indicator is a straight line bounded by two end icons.

3. The medical device holder of claim 1, wherein the base structure is a plate and the one or more transducer holders are integrally formed with the plate.

4. The medical device holder of claim 3, wherein the plate is bounded by two side edges, a bottom edge, and a concave arcuate top edge.

5. The medical device holder of claim 3, further comprising a belt coupled to the plate and configured to retain the plate in a position relative to a patient.

6. The medical device holder of claim 5, wherein the belt comprises a primary strap and a stabilizing strap.

7. The medical device holder of claim 6, wherein the primary strap is fixedly coupled to the plate at a first end of the primary strap.

8. The medical device holder of claim 6, wherein the stabilizing strap is fixedly coupled to the primary strap at a first end of the stabilizing strap.

9. The medical device holder of claim 8, wherein the stabilizing strap is releasably couplable to the plate at an opposing second end of the stabilizing strap.

10. The medical device holder of claim 9, wherein the primary strap is fixedly coupled to the plate at a first end of the primary strap and releasably couplable to the plate at an opposing second end of the primary strap.

11. The medical device holder of claim 1, wherein the base structure is a patch and the one or more transducer holders are coupled to the patch, and wherein the mid-line indicator extends from one end of the patch to an opposing end of the patch.

12. The medical device holder of claim 1, further comprising a holder panel coupled to the base structure, wherein the one or more transducer holders are coupled to the holder panel, and wherein the mid-line indicator extends from one end of the holder panel to an opposing end of the holder panel.

13. The medical device holder of claim 1, wherein each transducer holder respectively comprises two wing panels configured to receive and retain lateral wings of a transducer therein.

14. The medical device holder of claim 1, wherein the base structure is a belt configured to be wrapped around a chest of the patient.

15. The medical device holder of claim 1, wherein the base structure is a patch configured to be applied to skin of the patient.

16. The medical device holder of claim 15, wherein the patch is a transparent patch.

17. The medical device holder of claim 1, wherein the one or more transducers include at least two transducers in a longitudinally aligned configuration.

18. The medical device holder of claim 1, wherein the one or more transducers include at least two transducers arranged symmetrically across the mid-line indicator.

19. The medical device holder of claim 1, wherein the one or more transducers include two transducers in a laterally aligned configuration and at least a third transducer longitudinally offset from the two transducers.

20. The medical device holder of claim 1, wherein the mid-line indicator is two end icons adjacent two opposite edges of the base structure.
